(19) United States
(12) Patent Application Publication
(10) Pub. No.: US 2011/0045461 A1
(43) Pub. Date: Feb. 24, 2011

(54) USE OF ANTI-MICROBIAL PEPTIDES AS BACTERIAL INFECTION RESISTANCE MARKERS IN PENAEOID SHRIMP

(76) Inventors: Julien De Lorgeril, Montpellier (FR); Evelyne Bachere, Clapiers (FR); Yannick Gueguen, Jacou (FR); David Piquemal, Martin De Valgalgues (FR)

Correspondence Address: MORGAN LEWIS & BOCKIUS LLP, 1111 PENNSYLVANIA AVENUE NW, WASHINGTON, DC, 20004, US

(21) Appl. No.: 12/513,863

(22) PCT Filed: Nov. 6, 2007
(86) PCT No.: PCT/FR07/01830

§ 371 Date: Nov. 2, 2010

(30) Foreign Application Priority Data
Nov. 7, 2006 (FR) FR 0609724

(51) Int. Cl. C12Q 1/68

(52) U.S. Cl. 435/6

(57) ABSTRACT

The present invention relates to the use of transcripts of genes encoding antimicrobial peptides, for evaluating the resistance of penaeid shrimp, in particular of the species Litopenaeus stylirostris, to bacterial infections, in particular to vibriosis.

The present invention relates to the use of transcripts of genes encoding antimicrobial peptides as markers for the resistance of penaeid shrimp to bacterial infections.

The farming of penaeid shrimp in aquaculture involves many countries in tropical, subtropical and temperate zones. Nine species of penaeid shrimp are mainly used: Litopenaeus vannamei (Pacific white shrimp), which is the species most widely used, L. stylirostris (Pacific blue shrimp), three species of Atlantic origin, including L. setiferus (Atlantic white shrimp), L. schmitti (southern white shrimp) and Farfanpenaeus paulensis (Sao Paulo shrimp), and also Penaeus semisulcatus (green tiger prawn), P. monodon (black tiger prawn), Fenneropenaeus chinensis (fleshy prawn) and Marsupenaeus japonicus (Kuruma prawn).

A major problem for this aquaculture is caused by infectious diseases, predominantly of viral or bacterial origin.

As regards the bacterial pathologies, the main agents described belong to the Vibrionaceae genus (VANDENBERGHE et al., Appl. Environ. Microbiol., 65: 2592-7, 1999). Although Vibrios are generally considered to be commensal or symbiotic bacteria, many species can become pathogenic for shrimp under certain environmental conditions. Among the most widely studied, mention will be made of Vibrio penaeicida, which is pathogenic for the larvae and also for the juvenile and adult shrimp, and which is commonly used as a model of Vibrio infection in shrimp.

This pathogenic Vibrio is, moreover, the principal etiological agent associated with cyclic mortalities called “syndrome 93”, which appeared in 1993 and which affect New Calcdonian hatcheries during the growth phase (MERMOUD et al., Aquaculture, 323-335, 1998).

Antibiotics have been very widely used as preventive and curative agents in the treatment of the animals in the water of hatcheries. Their use is today called into question because of the appearance of resistant bacteria, and also because of the environmental imbalances that they may create.

New prophylactic methods have been proposed for controlling diseases, for instance the use of probiotic bacteria which would have beneficial effects on the survival and the growth of the animals in hatcheries, or that of immunostimulants, such as β-glucans or poly-saccharides derived from yeasts or from bacteria.

The vaccination methods used in vertebrates cannot be envisaged in shellfish because of the absence of an immune system with a memory. In shellfish, the defence against infections is based on an innate immune response, the principal participants of which are the haemocytes (blood cells), which in particular produce various antimicrobial peptides.

In penaeid shrimp, the antimicrobial peptides most well known to date belong to the penaeidin family (BACHERE et al., Immunol., Rev., 198: 149-68, 2004). The penaeidins identified at the current time have been classified into 3 subgroups, respectively called PEN2, PEN3 and PEN4 (GUEGUEN et al., Dev. Comp. Immunol., 30: 283-8, 2006), the PEN3 subgroup being the most represented in quantitative terms.

Other antimicrobial polypeptides have also been identified in shrimp. By way of examples, mention will be made of polypeptides that are homologues of horseshoe crab anti-lipopolysaccharide factors (ALFs), identified from P. monodon haemocyte cDNA libraries (SOMBOONWIWAT et al., Dev. Comp. Immunol., 29: 841-51, 2005), and also homologues of lysozyme and of an antimicrobial protein of 11.5 kDa, called crustin, identified in ESTs of various species of penaeids (GROSS et al., Dev. Comp. Immunol., 25: 565-77, 2001; SUPUNGUL et al., Mar. Biotechnol. (NY), 4: 487-94, 2002).

In penaeid shrimp, the study of the haemocyte population, and the study of penaeidin expression, in response to stimulation with nonpathogenic agents, or during infection with pathogenic bacteria (MUNOZ et al., Eur. J. Biochem., 269: 2678-89, 2002; MUNOZ et al., Cell Mol. Life. Sci., 61: 961-72, 2004; DESTOUMIEUX et al., J. Cell Sci., 113(Pt 3): 461-9, 2000), have made it possible to gain a better understanding of the mechanisms of the immune response.

The first phase of the immune response of penaeids to a bacterial infection, corresponding to the first 6 or 12 hours post-infection, is characterized by a decrease in the total number of circulating haemocytes, and also by a decrease in the relative expression of penaeidins. This decrease reflects migration of the haemocytes to the sites of infection, where they are lysed, and release the penaeidins.

This first phase is followed by a return to a basal level 24 to 48 hours after the infection.

During the second phase of the immune response, corresponding to 48-72 hours post-infection, the released penaeidins are observed both in the tissues and in the plasma. Moreover, a large increase in haemocytes expressing penaeidins is observed both in the blood stream and in most of the tissues. This increase in haemocytes reflects a process of haematopoiesis activation, producing a new population of granular haemocytes, characterized by a penaeidin transcription activity that is greater than that of the mature haemocytes.

The genetic selection of infection-resistant animal lines constitutes a potentially advantageous approach for perpetuating shrimp productions. In order to implement this approach, it is desirable to have markers for evaluating or predicting the level of resistance of the animals to infection.

In prior studies (DE LORGERIL et al., Physiol. Genomics, 21: 174-83, 2005), the inventors' team studied, by suppression subtractive hybridization (SSH), the expression of various genes before and during an experimental infection with V. penaeicida, in shrimp which survive this infection.

They thus identified various genes among which are, in particular, genes encoding antimicrobial peptides, such as penaeidin-2 (Litsty PEN2) and penaeidin-3 (Litsty PEN3), lysozyme, and also a cryptdin-like peptide rich in proline and cysteine. On the other hand, the genes encoding other antimicrobial peptides normally expressed in shrimp haemocytes, i.e. crustins and the anti-LPS factor (ALF), were not identified in this SSH library.

The level of transcription of these genes during the infection was also studied in the shrimp which survive the infection, and in those which did not. The level of transcription of the genes encoding penaeidin-3, lysozyme and the peptide rich in proline and cysteine decreases at the beginning of infection in all the animals, and then subsequently increases only in the animals which survive the infection. It is concluded from this that the profiles of differential expression of these 3 genes during the infection may constitute good markers for evaluating the ability of shrimp to survive infection.

The inventors undertook to search for other markers that can be used to evaluate the immune response of shrimp to infections and the survival thereof with respect to infections.

These studies were carried out on two lines of shrimp of the species Litopenaeus stylirostris: one, which is highly resistant to vibriosis, is a line obtained in the third generation from breeder shrimp having survived infectious episodes of “syndrome 93”, the other, referred to as “control” or “nonselected” line, is derived from animals farmed under the farming conditions commonly used in New Calcdonia. They were subsequently carried out on a population of animals farmed in Polynesia, and having undergone no selection with respect to their resistance to infections.

The inventors thus demonstrated other genes of which the profile of transcription in response to infection with a Vibrio is correlated with survival in response to this infection, and have in particular discovered that, in the case of some genes, the basal level of transcription (i.e. the level of expression in the absence of infection with a pathogen) is also correlated with survival in response to the infection.

The present invention concerns the use of these various genes as predictive markers for the resistance of penaeid shrimp to infections, in particular to vibriosis.

The inventors have observed that, in the shrimp of the line selected for its vibriosis resistance, the average basal level of transcription of lysozyme and of the PEN3-1 isoform is greater than that of the control line, whereas that of the PEN3-2 isoform is less than that of the control line. The experiments carried out on the population of nonselected animals have made it possible to note that the average basal level of transcription of the PEN 3-1 isoform, of penaeidin-2 (PEN 2) and of ALF is higher in the animals which survive the infection than in those which do not survive said infection. The inventors have also demonstrated a new gene, encoding a peptide hereinafter referred to as “glycine-rich peptide”, and have noted that the average basal level of transcription of this gene is lower in the animals which survive the infection than in those which do not survive the infection.

The cDNA sequence of the L. stylirostris lysozyme transcript is represented in the sequence listing in the annex under the number SEQ ID NO: 1; it is also available on GenBank under accession No. CV699332.

Homologues of the sequence SEQ ID NO: 1 in other shrimp species are available on GenBank under the following accession numbers: Litopenaeus vannamei (AF425673), Penaeus monodon (AF539466), Fenneropenaeus chinensis (AY661543), Penaeus semisulcatus (AY169675), and Marsupenaeus japonicus (AB080238).

The cDNA sequence of the L. stylirostris PEN3-1 transcript is represented in the sequence listing in the annex under the number SEQ ID NO: 2; it corresponds to that found in the genomic DNA (GenBank accession number AY351656 for L. stylirostris).

The cDNA sequence of the L. stylirostris PEN3-2 transcript is represented in the sequence listing in the annex under the number SEQ ID NO: 3; it derives from post-transcriptional modifications of PEN3-1, involving RNA editing, and also the removal of a motif of a few amino acids.

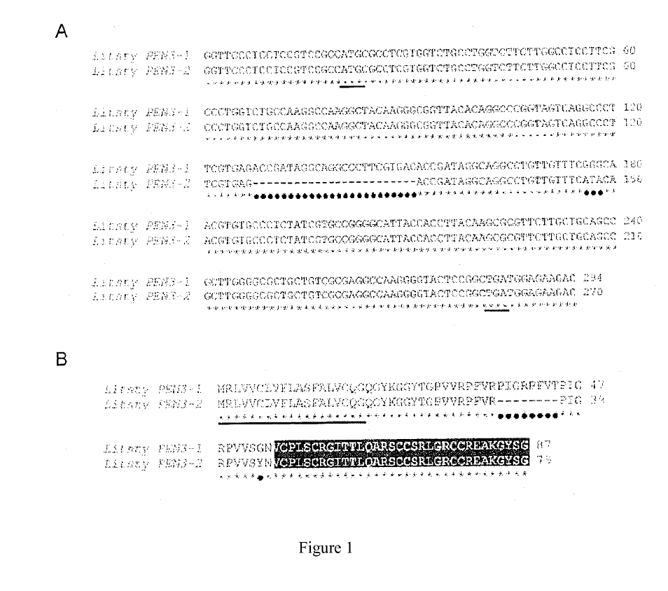

The comparison between the sequences of these two isoforms is illustrated by FIG. 1.

The identical regions are indicated by asterisks, the dissimilar regions by dots. In the nucleotide sequences, the translation start and stop codons are underlined. In the polypeptide sequences (SEQ ID NOs: 38 and 39), the signal peptides are underlined, the regions rich in glycine and proline are highlighted in light grey, and the regions rich in cysteines in dark grey.

The cDNA sequence of L. stylirostris penaeidin-2 (called PEN2, or PEN2-1) is available on GenBank under accession number AY351655. It is also represented in the sequence listing in the annex under the number SEQ ID NO: 45.

The cDNA sequence of the L. stylirostris anti-LPS factor (ALF) is represented in the sequence listing in the annex under the number SEQ ID NO: 40.

The cDNA sequence of the L. stylirostris glycine-rich peptide is represented in the sequence listing in the annex under the number SEQ ID NO: 42.

A subject of the present invention is a method for evaluating the capacity of resistance, of a population of penaeid shrimp, to infection with a pathogenic bacterium, characterized in that it comprises the quantification of the mRNA of the PEN3-1 isoform of penaeidin-3 in haemocytes taken from a sample of noninfected animals of the test population.

According to one preferred embodiment of the method in accordance with the invention, it also comprises the quantification of the mRNA of the PEN3-2 isoform of penaeidin-3, and the determination of the PEN3-1 isoform/PEN3-2 isoform ratio.

PEN3-1 isoform/PEN3-2 isoform ratios greater than 1, preferably greater than 2, and entirely preferably greater than 3, are predictive of a high capacity of resistance to infection.

Advantageously, the method in accordance with the invention also comprises the quantification of at least one of the following RNAs:
the penaeidin-2 (PEN-2) mRNA;
the glycine-rich-protein mRNA;
the ALF mRNA.

A subject of the present invention is also a method for selecting the animals having the best capacity of resistance to infection with a pathogenic bacterium in a population of penaeid shrimp, characterized in that it comprises the quantification of the mRNA of the PEN3-1 isoform of penaeidin-3 in haemocytes taken from a sample of noninfected animals of the test population, and the selection of the animals in which the level of expression of said mRNA is the highest.

According to one preferred embodiment of this method, it also comprises the quantification of the mRNA of the PEN3-2 isoform of penaeidin-3, the determination of the PEN3-1 isoform/PEN3-2 isoform ratio, and the selection of animals in which this ratio is the highest.

Advantageously, the method in accordance with the invention also comprises the quantification of at least one of the following RNAs:
the penaeidin-2 (PEN-2) mRNA;
the glycine-rich-protein mRNA;
the ALF mRNA,

and the selection of the animals in which the level of expression of the penaeidin-2 mRNA and/or the level of expression of the ALF mRNA is the highest and/or the level of expression of the glycine-rich-protein mRNA is the lowest.

According to one variant of the method in accordance with the invention, it is used to evaluate the capacity of resistance, of a line of penaeid shrimp, to infection with a pathogenic bacterium, and it comprises, in addition to the quantification of the mRNA of the PEN3-1 isoform and, advantageously, of the mRNA of the PEN3-2 isoform of penaeidin-3, in haemocytes taken from noninfected animals of the test line, the quantification of at least one of the following RNAs:
the lysozyme mRNA;
the penaeidin-3 mRNA.

The term “penaeidin-3 mRNA” defines herein all the transcripts of the isoforms of this gene.

According to one preferred embodiment of this variant of the method in accordance with the invention, the amount of one or more of the mRNAs defined above is determined in the test animals and is compared with the average amount of the same mRNA in noninfected animals of a control line (i.e. a line not selected for its resistance to a pathogenic bacterium) of shrimp of the same species.

In the case of the lysozyme, or of the PEN3-1 isoform, a transcript level greater than that of the control shrimp is predictive of a high capacity of resistance to infection. In the case of penaeidin-3, or of the PEN3-2 isoform, a transcript level lower than that of the control shrimp is predictive of a high capacity of resistance to infection.

According to another preferred embodiment of this variant of the method in accordance with the invention, it comprises the comparison between the amounts of mRNA of the two members of at least one of the following pairs:
lysozyme/penaeidin-3;
lysozyme/PEN3-2 isoform;
PEN3-1 isoform/penaeidin-3;
PEN3-1 isoform/PEN3-2 isoform.

A lysozyme/penaeidin-3 ratio above 1.2, preferably above 6.34, in the same way as a lysozyme/PEN3-2 isoform ratio above 0.077, preferably above 1.02, a PEN3-1 isoform/penaeidin-3 ratio above 23.09, preferably above 46.87, or a PEN3-1 isoform/PEN3-2 isoform ratio above 1.48, preferably above 7.7, is predictive of a high capacity of resistance to infection.

The present invention also makes it possible to evaluate the outcome of an infection with a pathogenic bacterium in a penaeid shrimp, on the basis of the profiles of transcription of various genes over the course of the infection.

The profiles of transcription over the course of the infection which appear to be correlated with the outcome of the infection are, in addition to those of penaeidin-3, of lysozyme and of the peptide rich in proline and cysteine, previously identified (DE LORGERIL et al., 2005, mentioned above), those of penaeidin-2, of crustin and of the PEN3-1 isoform.

In all cases, said transcription profiles are observed 6 to 72 hours after infection, preferably 24 hours after infection.

After infection, in the case of crustin and of the peptide rich in proline and cysteine, a higher level of transcription is observed in the animals which survive the infection than in those which do not survive it. In the case of crustin, this level is substantially equal to the basal level of transcription (measured prior to the infection) in the animals which survive, whereas it is significantly lower in those which do not survive. In the case of the peptide rich in proline and cysteine, this level is slightly lower than the basal level of transcription in the animals which survive, whereas it is significantly lower in those which do not survive.

For the other genes, the correlation between the gene expression profile and the resistance of the animal to infection depends on the line from which the individual tested is derived (control line or line selected for its resistance to infection).

In the case of lysozyme, of penaeidin-3 and of the PEN3-1 isoform:
for the control line, the level of transcription after the infection is higher in the surviving shrimp than in the nonsurviving shrimp; for lysozyme, it is equal to or higher than the basal level of transcription in the animals which survive, whereas it is significantly lower in those which do not survive; for penaeidin-3, it is slightly lower than the basal level of transcription in the animals which survive, whereas it is significantly lower in those which do not survive; for the PEN3-1 isoform, it is equal to or higher than the basal transcript level in the animals which survive, whereas it is significantly lower in those which do not survive;
for the selected line, the level of transcription after the infection does not significantly differ between the surviving shrimp and the nonsurviving shrimp.

In the case of penaeidin-2:

for the control line, the level of transcription after the infection does not significantly differ between the surviving shrimp and the nonsurviving shrimp;
for the selected line, the level of transcription after the infection is higher in the surviving shrimp than in the nonsurviving shrimp; it is not significantly different from the basal level of transcription in the animals which survive, whereas it is significantly lower (at most equal to 60% of the basal level) in those which do not survive.

A subject of the present invention is a method for evaluating the outcome of an infection with a pathogenic bacterium in a penaeid shrimp, characterized in that it comprises the quantification of the crustin mRNA, in haemocytes taken from the test animal 6 to 72 hours, preferably 24 hours, after the infection.

According to one preferred embodiment of a method in accordance with the invention, it also comprises the comparison of the amount of crustin mRNA determined as indicated above, with the amount of the same mRNA in haemocytes taken from the test animal prior to the infection.

The crustin cDNA sequence is represented in the sequence listing in the annex under the number SEQ ID NO: 4.

Homologues of the sequence SEQ ID NO: 4 in other shrimp species are available on GenBank under the following accession numbers: Litopenaeus vannamei (AF430076), Penaeus monodon (DWO42799), Maruspenaeus japonicus (AB121740) and Litopenaeus setiferus (AF430079).

A “crustin mRNA after infection/crustin mRNA before infection” ratio of between 0.9 and 1.1 is predictive of survival in response to infection; conversely, a “crustin mRNA after infection/crustin mRNA before infection” ratio of less than or equal to 0.6 is predictive of nonsurvival in response to infection.

Where appropriate, said method also comprises the quantification of the mRNA of the peptide rich in proline and in cysteine, in haemocytes taken from the test animal 6 to 72 hours, preferably 24 hours, after the infection, and, advantageously, the comparison of the amount of mRNA thus determined with the amount of the same mRNA in haemocytes taken from the test animal prior to the infection.

The cDNA sequence of the peptide rich in proline and cysteine is represented in the sequence listing in the annex under the number SEQ ID NO: 5.

Homologues of the sequence SEQ ID NO: 5 in other shrimp species are available on GenBank under the following accession numbers: Litopenaeus vannamei (BE188497) and Penaeus monodon (DT366712).

A “mRNA of the peptide rich in proline and cysteine after infection/mRNA of the peptide rich in proline and cysteine before infection” ratio of greater than or equal to 0.7 is predictive of survival in response to infection; conversely, a “mRNA of the peptide rich in proline and cysteine after infection/mRNA of the peptide rich in proline and cysteine before infection” ratio of less than or equal to 0.3 is predictive of nonsurvival in response to infection.

When the animals in which it is desired to evaluate the outcome of the infection do not belong to a line selected for its resistance to a pathogenic bacterium, it is also possible to quantify one or more of the following mRNAs:
the lysozyme mRNA;
the penaeidin-3 mRNA;
the mRNA of the PEN3-1 isoform of penaeidin-3,

in haemocytes taken from the test animal 6 to 72 hours, preferably 24 hours, after the infection, and, advantageously, to compare, for each mRNA chosen, the amount thus determined with that of the same mRNA in haemocytes taken from the test animal prior to the infection.

A “lysozyme mRNA after infection/lysozyme mRNA before infection” ratio of greater than or equal to 1 is predictive of survival in response to infection;

conversely, a “lysozyme mRNA after infection/lysozyme mRNA before infection” ratio of less than 0.2 is predictive of nonsurvival in response to infection.

A “penaeidin-3 mRNA after infection/penaeidin-3 mRNA before infection” ratio of greater than or equal to 0.8 is predictive of survival in response to infection; conversely, a “penaeidin-3 mRNA after infection/penaeidin-3 mRNA before infection” ratio of less than or equal to 0.5 is predictive of nonsurvival in response to infection.

A “PEN3-1 isoform mRNA after infection/PEN3-1 isoform mRNA before infection” ratio of greater than or equal to 1 is predictive of survival in response to infection; conversely, a “PEN3-1 isoform mRNA after infection/PEN3-1 isoform mRNA before infection” ratio of less than or equal to 0.2 is predictive of nonsurvival in response to infection.

Alternatively, when the animals in which it is desired to evaluate the outcome of the infection belong to a line selected for its resistance to a pathogenic bacterium, it is also possible to quantify the penaeidin-2 mRNA, in haemocytes taken from the test animal 6 to 72 hours, preferably 24 hour, after infection, and, advantageously, to compare the amount thus determined with that of the same mRNA in haemocytes taken from the test animal prior to the infection.

A “penaeidin-2 mRNA after infection/penaeidin-2 mRNA before infection” ratio of greater than or equal to 0.8 is predictive of survival in response to infection; conversely, a “penaeidin-2 mRNA after infection/penaeidin-2 mRNA before infection” ratio of less than or equal to 0.6 is predictive of nonsurvival in response to infection.

The methods in accordance with the invention can in particular be carried out in order to evaluate the resistance of penaeid shrimp, and in particular of L. stylirostris, to vibriosis, and in particular to infections with Vibrio penaeicida.

A large variety of methods for quantifying RNA, known in themselves to those skilled in the art, can be used for implementing the present invention (for review, cf., for example, QUERE et al., J. Virol. Methods, 105(2): 189-196, 2002; Patent FR 2 815 043 in the name of SKULD TECH; HUGGETT et al., Genes Immun., 6(4): 279-284, 2005; EMRICH et al., Methods Mol. Biol., 191: 99-108, 2002; POWELL, Methods Mol. Biol., 99: 297-319, 2000; NESS, Methods Mol. Biol., 316: 13-33, 2006; SCHENA et al., Trends Biotechnol., 16(7): 301-306, 1998; RAMSAY, Nat. Biotechnol., 16(1): 40-44, 1998).

By way of illustration, mention will be made of the various methods based on quantitative RT-PCR (reverse transcription followed by polymerase chain reaction). The general principle of these methods consists in selectively amplifying, by PCR, the gene(s) for which it is desired to measure the expression (target genes), from a cDNA preparation obtained by reverse transcription of the mRNAs extracted from the biological sample to be tested, and in quantifying the amplification product(s) obtained. The quantitative RT-PCR methods most widely used at the current time implement real-time PCR techniques (for review, cf., for example, VALASEK & REPA, Adv Physiol Educ, 29: 151-9, 2005), where the quantification of the amplification products is carried out by measuring the fluorescence thereof as they form during the exponential phase of the amplification reaction.

A subject of the present invention is also kits of reagents for carrying out a method in accordance with the invention, in which the quantification of the transcripts is performed by quantitative RT-PCR.

Kits of reagents in accordance with the invention comprise, in addition to the conventional buffers and reagents necessary for the amplification reaction and for the detection of the amplification products, at least one pair of primers specific for each of the mRNAs to be assayed.

According to a first embodiment, a kit of reagents in accordance with the invention comprises a pair of primers specific for the mRNA of the PEN3-1 isoform of penaeidin-3, and a pair of primers specific for the mRNA of the PEN3-2 isoform of penaeidin-3.

It may also comprise one or more pairs of primers chosen from:
a pair of primers specific for the penaeidin-2 (PEN-2) mRNA;
a pair of primers specific for the glycine-rich-protein mRNA;
a pair of primers specific for the ALF mRNA;
a pair of primers specific for the lysozyme mRNA.

According to another embodiment, a kit of reagents in accordance with the invention comprises a pair of primers specific for the crustin mRNA and, advantageously, a pair of primers specific for the mRNA of the peptide rich in proline and cysteine. Preferably, said kit also comprises a pair of primers specific for the lysozyme mRNA and/or a pair of primers specific for the penaeidin-3 mRNA and/or a pair of primers specific for the PEN3-1 isoform mRNA.

A kit in accordance with the invention may also comprise all the pairs of primers mentioned above, thereby making it possible to use it for carrying out all the methods described herein.

For the implementation of the present invention, use may also be made of DNA chips—also known as biochips, microarrays, chip, etc., —(for review, cf., for example, LOCKHART & WINZELER, Nature, 405: 827-36, 2000).

The general principle of DNA chips consists in fixing, at various sites on the surface of a solid support (for example, a glass, plastic or nylon surface), probes consisting of polynucleotides complementary to the various genes of which it is desired to measure the expression (target genes). The mRNAs extracted from the biological sample to be tested (and generally converted to cDNA by reverse transcription) are labelled and brought into contact with the probes fixed on the support. Measuring the hybridization signals makes it possible to evaluate the amount of the target-gene mRNAs in the sample to be tested.

A subject of the present invention is also DNA chips for carrying out a method in accordance with the invention.

According to a first embodiment, a DNA chip in accordance with the invention comprises a probe specific for the mRNA of the PEN3-1 isoform of penaeidin-3, and a probe specific for the mRNA of the PEN3-2 isoform of penaeidin-3.

It may also comprise one or more probes chosen from:
a probe specific for the penaeidin-2 (PEN-2) mRNA;
a probe specific for the glycine-rich-protein mRNA;
a probe specific for the ALF mRNA;
a probe specific for the lysozyme mRNA.

According to another embodiment, a DNA chip in accordance with the invention comprises a probe specific for the crustin mRNA and, advantageously, a probe specific for the mRNA of the peptide rich in proline and cysteine. Preferably, said chip also comprises a probe specific for the lysozyme mRNA and/or a probe specific for the penaeidin-3 mRNA and/or a probe specific for the PEN3-1 isoform mRNA.

A DNA chip in accordance with the invention may also comprise all the probes mentioned above, thereby making it possible to use it to carry out all the methods described herein.

The present invention will be understood more clearly from the further description which follows, which refers to nonlimiting examples, illustrating the correlation between the level of transcription of antimicrobial peptides in the haemocytes of penaeid shrimp, and the resistance of said shrimp to infection with Vibrio penaeicida.

### EXAMPLES

### Materials and Methods

Animals:

L. stylirostris juveniles (20-30 g) derived from two shrimp lines were obtained from the IFREMER [French Research Institute for Exploitation of the Sea] Laboratory of New Calcdonia (Ifremer, BP2059, 98846 Noumea Cedex, New Calcdonia, France).

One of these lines, called “selected line”, results from the crossing of animals having survived “syndrome 93”, raised in tanks for the first 6 months of their life, under conditions favourable to infection with Vibrio penaeicida (initial density: 20-25 PL/m²); the surviving animals are then raised at lower densities (1-2 shrimps/m²) so as to reproduce at the age of 12 months.

The other line, called “control line”, results from the crossing of animals raised in tanks under “traditional” conditions (initial density: 2 PL/m²), where the occurrence of “syndrome 93” is very low.

In both lines, inbreeding was controlled by individually marking the animals in order to prevent mating of close parents.

The animals used in the experiments described hereinafter were taken from the 3rd generation of the selected line (G3-selection), and from the 3rd generation of the control line (G3-control).

Furthermore, L. stylirostris juveniles (20-30 g) were obtained from the IFREMER Laboratory in Tahiti. Unlike the shrimp from New Calcdonia which live in an environment where pathogenic bacteria are constantly present, these animals have never been in contact with shrimp pathogenic bacteria.

### Experimental Infections:

The Vibrio penaeicida infection experiments were carried out by immersing the shrimp for 2 hours in tanks of sea water containing 1×10³ CFU/ml of the V. penaeicida strain AM101, which corresponds to 20% of the lethal dose (LD20) (SAULNIER et al., Dis. Aquatic Org., 40: 109-115, 2000). The animals were subsequently rinsed with filtered sea water, and transferred into 100 l tanks. Noninfected animals were stored in a separate 100 l tank.

For the first type of experimental infection, used for the overall analyses, animals of each shrimp line (control and selected) were divided up into three groups (15 shrimp per group) placed in separate tanks. For each of the shrimp lines, samples of the haemolymph of the first group were collected, 24 hours before the experimental infection (−24) so as to constitute the noninfected control; samples of the haemolymph of the other two groups were collected 12 hours after the infection (+12) and 24 hours after the infection (+24).

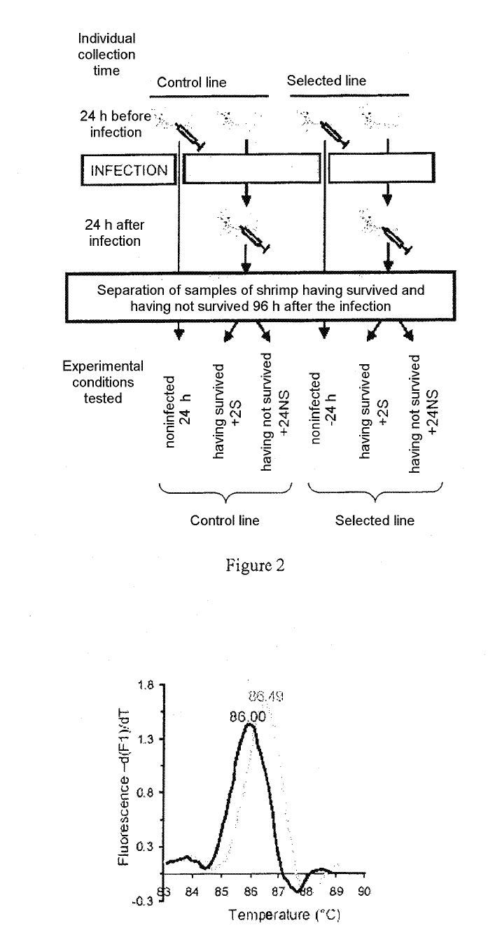

The protocol of the second type of experimental infection, used for the individual analyses, is illustrated by FIG. 2.

The shrimp are individually labelled by injecting coloured silicone under the third abdominal segment of the cuticle.

A first haemolymph sample is taken 24 hours before the infection, and a second sample is taken 24 hours after the infection.

The haemolymph samples from the animals which died during the 96 hours following the infection (acute mortality period), and those from the animals having survived at the end of this period, were individually distinguished.

The infections with Vibrio nigripulchritudo were carried out by injecting each shrimp with a solution containing “100 Vibrio nigripulchritudo bacteria” (60% lethal dose), 24 hours after labelling each individual of the shrimp population by injecting coloured silicone into an abdominal segment.

### RNA Isolation and Real-Time PCR Analysis:

The total RNA was isolated from the haemocytes of the shrimp using the Trizol reagent (Gibco BRL) (1 ml/10⁷ cells), according to the protocol indicated by the manufacturer.

The total RNA was treated with Dnase (TURBO DNase, Ambion) in order to eliminate the contaminating genomic DNA, and then the Dnase was eliminated by extraction with phenol-chloroform. The cDNA was synthesized from 1 μg of total RNA, using the SuperScript II reverse transcription kit, according to the instructions of the manufacturer (Invitrogen), in a reaction volume of 20 μl.

0.5 μl of each reverse transcription reaction was used as template for the real-time PCR in 10 μl of reaction volume containing 1×SYBR GREEN MASTER MIX (Qiagen) and 0.5 μM of each primer used.

Each real-time PCR reaction was carried out with an initial denaturation step of 900 s at 95° C., followed by amplification of the target cDNA (35 cycles of denaturation at 95° C. for 15 seconds, of hybridization between 54° C. and 64° C. (depending on the primer used) for 15 seconds, and of extension at 72° C. for 15 seconds), with the LightCycler (Roche Molecular Biomedicals). Each reaction was carried out in triplicate.

In order to determine the efficiency of each pair of primers used, standard curves were obtained using series of 5 dilutions of the plasmids containing the corresponding target sequences (10³ to 10⁷ copies/μl).

The real-time PCR results are represented in the form of variations in relative expression standardized with respect to a reference gene, elongation factor 1α (EF-1α, SEQ ID NO: 41), as described by PFAFFL (Nucleic Acids Res., 29: 45, 2001). For the genes tested, the real-time PCR efficiency ranges between 1.87 and 1.98. Since this efficiency is not exactly equal to 2.00 (representing 100% amplification efficiency at each cycle), the relative abundance was calculated, as indicated by PFAFFL (mentioned above, 2001), using the equation corrected for the differences in efficiency.

### Example 1

### Isolation of a New Penaeidin Sequence from The Pen3 Subgroup in the Haematocytes of L. stylirostris

A first real-time PCR series was carried out using the cDNAs of pools of haematocytes of 15 shrimp of each of the two lines, in order to compare the abundance of the transcript of the Litsty PEN3 peptide (GenBank AY351656) between these two lines, using the following primers:

[5′-GGTTCCCTCCTCCGTCCGCC-3′ (SEQ ID NO: 6) and 5′-GTCTTCTCCATCAGCCGGAG-3′ (SEQ ID NO: 7), efficiency 1.94, hybridization temperature 60° C.]

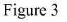

The analyses of the melting curves for the PCR products are shown in FIG. 3:

Legend of FIG. 3:

[symbol] selected shrimp line;

[symbol] control shrimp line.

The melting temperature corresponding for each peak is indicated above the peak.
The results show two melting peaks which vary by almost 0.5° C. The melting temperature of the PCR products appears to be higher for the selected shrimp line than for the control shrimp line (86.43±0.06 and 85.98±0.02, respectively, p<0.01, LightCycler 3.5 software).

The real-time PCR products having various melting temperatures were cloned in sequence, which led to the characterization of two cDNA sequences of 294 and 270 nucleotides (FIG. 1A). The sequence corresponding to the highest melting temperature, identified in the selected shrimp line, was identified as the Litsty PEN3 sequence previously characterized, and which will hereinafter be referred to as Litsty PEN3-1. The second cDNA sequence identified, in the control shrimp line, differs from Litsty PEN3-1 by virtue of a deletion of 24 nucleotides, and a change of three nucleotides in another part of the sequence (FIG. 1A). This sequence will hereinafter be referred to as Litsty PEN3-2.

At the amino acid sequence level, Litsty PEN3-2 differs from Litsty PEN3-1 by virtue of the absence, in the N-terminal domain, of 8 residues PIGRPFVT, and by virtue of the substitution of a glycine residue with a tyrosine residue (FIG. 1B).

Due to the close similarity between the Litsty PEN3-1 and Litsty PEN3-2 sequences, an investigation was carried out to determine whether they are expressed by two distinct genes or by a single gene. For this, the two sequences, i.e. the genomic DNA sequence and the cDNA sequence, of the haemocyte were amplified by PCR.

Pairs of primers for distinguishing these two sequences were constructed: P3-1R and P3-1F: [5′-ACCGATAGGCAGGCCCTTCGTGAC-3′ (SEQ ID NO: 8) and 5′-CGATAGAGGGCACACGTTGCCC-3′ (SEQ ID NO: 9), efficiency 1.85, hybridization temperature 64° C.] for Litsty PEN3-1 and P3-2R and P3-2F: [5′-GAGACCGATAGGCAGGCCTG-3′ (SEQ ID NO: 10) and 5′-CGATAGAGGGCACACGTTGTAT-3′ (SEQ ID NO: 11), efficiency 1.91, hybridization temperature 64° C.] for Litsty PEN3-2.

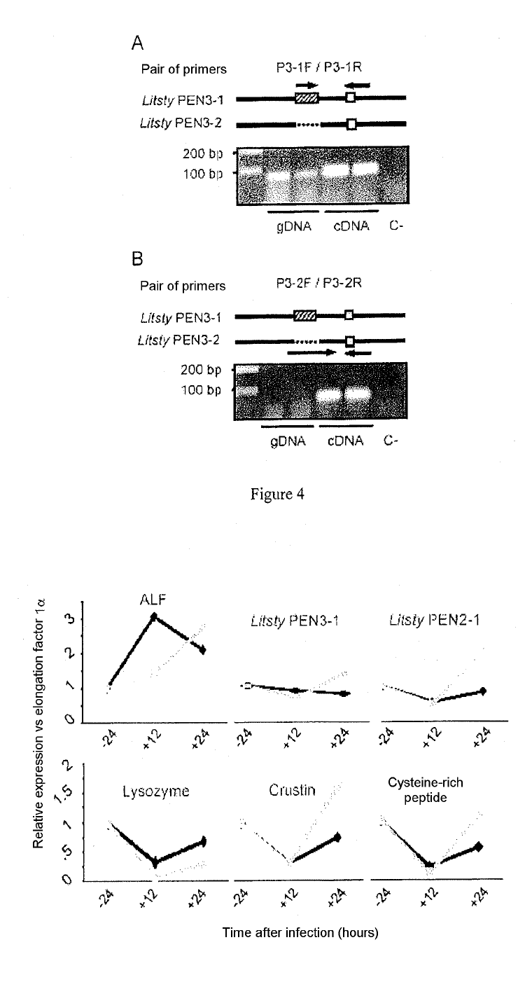

The results of agarose gel electrophoresis of the PCR products obtained with these primers for Litsty PEN3-1 and Litsty PEN3-2 are shown in FIG. 4A and FIG. 4B, respectively.

Legend of FIG. 4:

gDNA=genomic DNA
C−=negative control.

The pairs of primers used are indicated above the schematic representations of the two transcripts by arrows.

The nucleotide differences between the two transcripts are represented, for the deletion of 24 nucleotides, by a hatched rectangle on Litsty PEN3-1 and by a dotted line on Litsty PEN3-2, and, for the 3-nucleotide change, by a grey square on Litsty PEN3-1 and by a white square on Litsty PEN3-2.

Whatever the pairs of primers used,

Litsty PEN3-1 could be amplified from the genomic DNA and from the cDNA. The amplified fragments are of the same size in both cases, indicating the absence of introns (FIG. 4A). In the case of Litsty PEN3-2, the amplification is obtained only from cDNA, and not from the genomic DNA (FIG. 4B).

### Example 2

### Antimicrobial-Peptide Gene Expression Profiles Over the Course of an Infection with Vibrio penaeicida

In order to analyse the antimicrobial peptide (AMP) gene expression in the circulating haemocytes of shrimp, 6 cDNA sequences of AMPs (or putative AMPs) described in recent studies (MUNOZ et al., Cell. Mol. Life. Sci., 61: 961-972, 2004; DE LORGERIL et al., 2005, mentioned above) were studied in L. stylirostris. The GenBank accession numbers of the penaeidins Litsty PEN2-1 and PEN3 are, respectively, AY351655 and AY351656. The GenBank accession numbers of the lysozyme and of the “cysteine-rich peptide” are, respectively, CV699332 and CV699287. The crustin cDNA sequence is indicated in the sequence listing in the annex under the number SEQ ID NO: 4. The ALF cDNA sequence is indicated in the sequence listing in the annex under the number SEQ ID NO: 40.

The expression profiles for the genes encoding the AMPs, Litsty PEN2-1 and -3, ALF, crustin, lysozyme and cysteine-rich peptide were analysed by real-time PCR in order to examine the general expression scheme during the development of the infection with V. penaeicida, for the selected line or for the control line.

The PCR primers (sense and antisense) listed in Table 1 below were used. The hybridization temperature specific for each pair of primers is indicated, as is the efficiency of the PCR; calculated by the equation E=10[−1/slope] (PFAFFL, 2001, mentioned above).

TABLE 1

| Gene | Sense | Antisense | Hybrid- | PCR effic- |
|---|---|---|---|---|
| name | (5′→3′) primer | (5′→3′) primer | ization (° C.) | iency |
| ALF | GCCACTCCTGCACCTACAA | TTCACAACACCGGATTTGCTG | 55 | 1.87 |
| | (SEQ ID NO: 12) | (SEQ ID NO: 13) | | |
| Litsty | CCATGCGCCTCGTGGTCTG | GAACGCGCTTGTAAGGTGGTAA | 64 | 1.98 |
| PEN3 | (SEQ ID NO: 14) | (SEQ ID NO: 15) | | |
| Litsty | CCATGCGCCTCGTGGTCTG | AGCAATTGCGGCATCTGGGAA | 64 | 1.91 |
| PEN2-1 | (SEQ ID NO: 16) | (SEQ ID NO: 17) | | |
| Lysozyme | CGTCTGCACGTCAGCTGTG | GGCTTGGCACCAGGGTTACC | 59 | 1.93 |
| | (SEQ ID NO: 18) | (SEQ ID NO: 19) | | |
| Crustin | CCAAGTCCGTCCCACATGC | CTGTCGAAGCAGCACTTGTC | 55 | 1.91 |
| | (SEQ ID NO: 20) | (SEQ ID NO: 21) | | |
| Cysteine- | GCCCTAAGTGCCCATATGA | GCCGCATTCACATCCTATA | 54 | 1.89 |
| rich | (SEQ ID NO: 22) | (SEQ ID NO: 23) | | |
| peptide | | | | |
| EF-1α | GGTGCTGGACAAGCTGAAGGC | CGTTCCGGTGATCATGTTCTTGATG | 60 | 1.96 |
| | (SEQ ID NO: 24) | (SEQ ID NO: 25) | | |

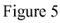

The results are given in FIG. 5. The relative expressions were standardized with respect to elongation factor 1α, and each value was calculated with reference to the noninfected shrimp (relative expression=1) according to the efficiency-corrected 2-ΔΔCt method (PFAFFL, 2001, mentioned above).

Legend of FIG. 5:

[symbol]

selected shrimp line; [symbol]
control shrimp line.
−24=noninfected shrimp
+12=12 hours after the infection
+24=24 hours after the infection.

It appears that the amount of the ALF, lysozyme, crustin and cysteine-rich-peptide RNAs varies during the infection, and compared with the noninfected shrimp, in the two shrimp lines.

Only ALF exhibits an expression scheme over the course of the infection which differs between the two shrimp lines. Specifically, the amount of ALF transcript increases starting from 12 hours after the infection for the control line (relative increase of, respectively, 3× and 2.1×, 12 and 24 hours after the infection), whereas the increase (by a factor of 2.7×) is delayed to 24 hours after the infection for the selected shrimp line.

For the other AMPs, the amount of transcripts decreases in the first 12 hours following the infection. This is particularly obvious for lysozyme, crustin and the cysteine-rich peptide, in the control line (relative decrease of 3.2×, 3.3× and 4.5× compared with the noninfected shrimp, respectively) just as in the selected line (relative decrease of 25×, 3.5× and 9.1× compared with the noninfected shrimp, respectively). The decrease in crustin transcripts and in cysteine-rich-peptide transcripts is followed, 24 hours after the infection, by a tendency to return to the levels observed in the noninfected shrimp. This tendency is observed for both shrimp lines, but the increase in crustin transcripts and in cysteine-rich-peptide transcripts between 12 and 24 hours appears to be significantly greater in the selected line (relative increase of 5.8× and 9.8×, respectively), than in the control line (relative increase of 2.5× and 2.6×, respectively).

The same decrease is observed for lysozyme 12 hours after the infection for both lines; however, the lysozyme transcripts remain less abundant, 24 hours after the infection, for the selected shrimp line than for the control shrimp line.

As regards the penaeidins Litsty PEN2-1 and Litsty PEN3, their expression varies only slightly in the control line, in particular for Litsty PEN3. In the selected line, the amount of Litsty PEN2-1 and Litsty PEN3 transcripts increases (by respectively 3.93× and 2.73×) between 12 hours and 24 hours after the infection.

### Example 3

### Demonstration of the Relationship Between The Basal Expression Level of Antimicrobial Peptides And the Resistance to Infection of a Shrimp Line

### Expression of Lysozyme, and Overall Expression of Penaeidin-3

The basal level of transcription of the lysozyme, of Litsty PEN3, and of the two isoforms Litsty PEN3-1 and Litsty PEN3-2 was compared in the two shrimp lines (selected and control). The amount of transcripts 24 hours before the infection was measured by real-time PCR, as indicated above in Materials and Methods, using pools of haemocyte RNA samples (pools of 15 shrimp) taken 24 hours before the infection.

In order to quantify the lysozyme transcription and the overall transcription of Litsty PEN3, the primers used are those indicated in Table 1 above. In order to specifically quantify the transcription of Litsty PEN3-1 and Litsty PEN3-2, the primers P3-1R and P3-1F for Litsty PEN3-1, and P3-2R and P3-2F for Litsty PEN3-2, described in Example 1, were used.

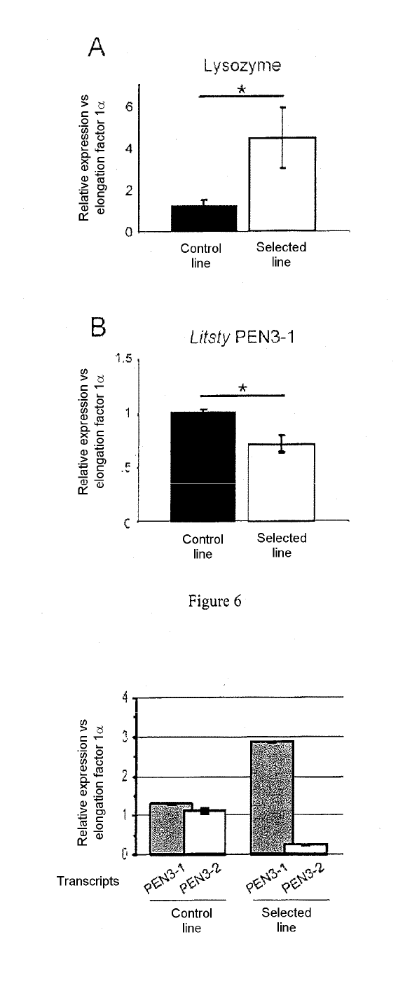

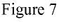

The results obtained for the lysozyme are illustrated by FIG. 6A, and those obtained for Litsty PEN3 are illustrated by FIG. 6B. The results obtained for Litsty PEN3-1 and Litsty PEN3-2 are given in FIG. 7.

The relative expression levels were standardized with respect to elongation factor 1α. In the case of the lysozyme and of Litsty PEN3 (FIG. 6), the results were expressed as mean values±SDV from 5 or 6 shrimp per shrimp line. Each value is calculated with reference to the group of the control line (relative expression=1) according to the efficiency-corrected 2-ΔΔCt method (PFAFFL, 2001, mentioned above). In the case of Litsty PEN3-1 and Litsty PEN3-2 (FIG. 7), the relative expression levels were standardized with respect to EF-1α, and each value was calculated with reference to the first value of Litsty PEN3-2 using the control line (relative expression=1) according to the efficiency-corrected 2-ΔΔCt method. The results are expressed as mean values±SDV from two pools of shrimp, determined in triplicate.

Legend of FIG. 6:

▪ control shrimp line
□ selected shrimp line
* statistical difference between the two shrimp lines, p<0.05 (Student's test)

Legend of FIG. 7:

[symbol]

Litsty PEN3-1 transcripts
□ Litsty PEN3-2 transcripts

These results reveal significant differences between the shrimp of the line selected for survival and the shrimp of the control line.

As regards the lysozyme, the basal level of transcription is lower in the haemocytes of the shrimp of the control line, than in those of the shrimp of the selected line.

Conversely, as regards the overall transcription of Litsty PEN3, its basal level is higher in the haemocytes of the shrimp of the control line than those of the shrimp of the selected line.

As regards Litsty PEN3-1, its basal level of transcription is much higher in the haemocytes of the shrimp of the selected line than in those of the shrimp of the control line.

Conversely, the basal level of transcription of Litsty PEN3-2 is much lower in the haemocytes of the shrimp of the selected line than in those of the shrimp of the control line.

In addition, in the selected line, the basal level of transcription of Litsty PEN3-1 is much higher than that of Litsty PEN3-2, whereas no difference between the basal levels of transcription of Litsty PEN3-1 and Litsty PEN3-2 can be observed in the control line.

### Example 4

### Relationship Between the Antimicrobial-Peptide Gene Expression Profile Over the Course of the Infection with Vibrio penaeicida and the Individual Survival Capacity of the Shrimp

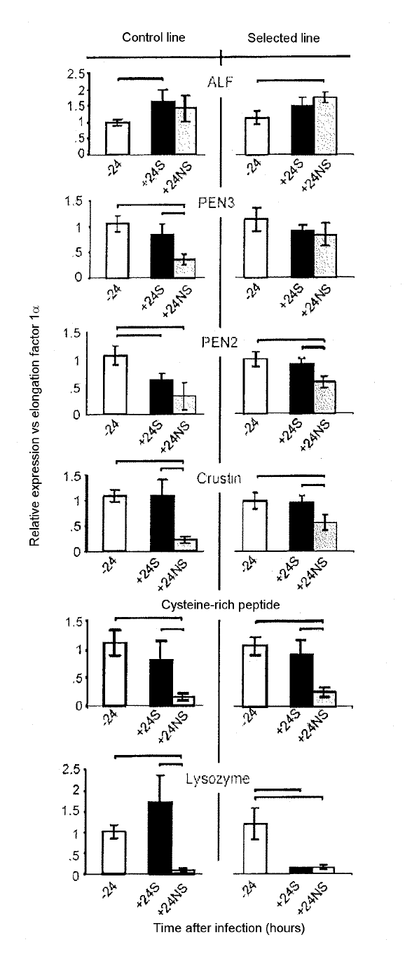

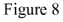

The amounts of ALF transcripts, of Litsty PEN3 (PEN3) transcripts and transcripts of its isoforms Litsty PEN3-1 and Litsty PEN3-2, of Litsty PEN2-1 (PEN2) transcripts, of lysozyme transcripts, of crustin and of cysteine-rich-peptide transcripts, 24 hours before the infection and 24 hours after the infection, were compared between the individuals having survived the infection and those having not survived, in the selected shrimp line and in the control line. The results are given in FIGS. 8 and 9.

The amount of transcripts was determined by real-time PCR, as described above, from individual samples (5 or 6 shrimp per condition), and the results given represent the mean of these measurements. The levels of relative expression were standardized with respect to EF-1α.

For ALF, Litsty PEN3, Litsty PEN2-1, lysozyme, crustin and the cysteine-rich peptide (FIG. 8), the values during the infection were calculated with reference to the noninfected group (relative expression=1) according to the efficiency-corrected 2-ΔΔCt method. The experimental conditions associated by a square bracket show a statistical difference, p<0.05

(ANOVA-test LSD).

Legend of FIG. 8:

□: amount before the infection (−24)
▪: amount 24 hours after the infection in the shrimp having survived (+24S) [symbol]
amount 24 hours after the infection in the shrimp having not survived (+24NS).

For ALF, an increase in the transcripts is observed 24 hours after the infection in the two shrimp lines. In the control line, a significant difference in the level of transcription compared with the noninfected shrimp is observed for the shrimp having survived, but not for those having not survived. Conversely, in the selected shrimp line, a significant difference in the level of transcription compared with the noninfected shrimp is observed for the shrimp having not survived, but not for those having survived. Neither of the two lines shows a significant difference between the shrimp having survived and those having not survived.

For the penaeidin family, the Litsty PEN2-1 and PEN3 transcripts decrease 24 hours after the infection.

For Litsty PEN3, significant differences in the levels of transcription are observed only in the control line, between the shrimp having not survived and the noninfected shrimp, and also between the shrimp having not survived and those having survived.

For Litsty PEN2-1, significant differences are observed in the two lines.

In the control line, a significant decrease in the transcripts compared with the noninfected shrimp is observed both in the shrimp having survived and in those having not survived.

In the selected line, significant differences are observed between the shrimp having survived and those having not survived, and also between the shrimp having not survived and the noninfected shrimp. On the other hand, no significant difference is observed between the noninfected shrimp and those having survived.

The crustin and cysteine-rich-peptide transcripts exhibit similar expression schemes in the two shrimp lines.

In both lines, a significant decrease in the crustin and the cysteine-rich-peptide transcripts is observed in the shrimp having not survived compared with the noninfected animals, and a significant increase in these transcripts is observed in the animals having survived compared with those having not survived.

In the case of the lysozyme, in the control line, a large decrease in the amount of transcripts is observed in the shrimp having not survived compared with the noninfected shrimp, and compared with the shrimp having survived. In the selected line, a significant decrease in the amount of transcripts is observed both in the shrimp having survived and in those having not survived, compared with the noninfected shrimp.

For Litsty PEN3-1 and Litsty PEN3-2 (FIG. 9), the values during the infection were calculated with reference to the mean of all the values according to the efficiency-corrected 2-ΔΔCt method.

Legend of FIG. 9:

[symbol]

relative expression of Litsty PEN3-1 transcripts (P3-1)
□ relative expression of Litsty PEN3-2 transcripts (P3-2)
−24=shrimp collected before the infection
+24S=shrimp, collected 24 hours after the infection, having survived
+24NS=shrimp, collected 24 hours after the infection, having not survived.

In the control line, significant differences are observed between the amount of Litsty PEN3-1 transcripts and the amount of Litsty PEN3-2 transcripts only 24 hours after the infection, and in the shrimp having survived. For the animals which have not survived, the amounts of the two transcripts Litsty PEN3-1 and Litsty PEN3-2 are extremely low compared with those measured before the infection.

In the selected line, significant differences are observed between the amounts of Litsty PEN3-1 and Litsty PEN3-2 irrespective of the time of the infection considered.

Litsty PEN3-1 is much more abundant than Litsty PEN3-2, whether before the infection, at 24 hours in the shrimp which have survived, or in those which have not survived.

### Example 5

### Analysis of the Transcript Expression on a DNA Chip

Miniarray membranes were prepared in order to evaluate the abundances of transcripts of genes encoding six antimicrobial peptides (ALF, crustin, glycine-rich peptide, Litsty PEN3-1, lysozyme, cysteine-rich peptide) of the shrimp L. stylirostris.

The GenBank accession numbers of the lysozyme and of the “cysteine-rich peptide” are, respectively, CV699332 and CV699287. The PEN3-1 and crustin cDNA sequences are, respectively, indicated in the sequence listing in the annex under the numbers SEQ ID NO: 2 and SEQ ID NO: 4. The ALF cDNA sequence is indicated in the sequence listing in the annex under the number SEQ ID NO: 40. The “glycine-rich-peptide” cDNA sequence is indicated in the sequence listing in the annex under the number SEQ ID NO: 42.

Preparation of Probe cDNAs

cDNA fragments (probe cDNA) of each of the genes encoding the six antimicrobial peptides are amplified by conventional PCR.

The PCR primers (sense and antisense) listed in Table 2 below were used. The hybridization temperatures specific for each pair of primers is indicated, as is the expected size of the PCR products amplified.

TABLE 2

| | Primer | | | Tm | Size |
|---|---|---|---|---|---|
| Gene name | name | Primer sequence | | (° C.) | (bp) |
| Cysteine- | CystF | 5′-TGGTGGCCGAACTCTGGAC-3′ | (SEQ ID NO: 26) | 62 | 475 |
| rich | CystR | 5′-CTCCTTTCTCCCATCTCACG-3′ | (SEQ ID NO: 27) | 62 | |
| peptide | | | | | |
| Glycine- | GlyF | 5′-TGCACCGAGCAAAACGTCAG-3′ | (SEQ ID NO: 28) | 62 | 449 |
| rich | GlyR | 5′-TCGAGGAATTCACACTGAAACA-3′ | (SEQ ID NO: 29) | 62 | |
| peptide | | | | | |
| Crustin | CrusF | 5′-GTGGTGGCGGTTTAGGTGTA-3′ | (SEQ ID NO: 30) | 62 | 429 |
| | CrusR | 5′-CATCGGTCGTCCTTCAGATG-3′ | (SEQ ID NO: 31) | 62 | |
| Lysozyme | LysoF | 5′-TATCAACCATTACAACGTCTGC-3′ | (SEQ ID NO: 32) | 62 | 201 |
| | LysoR | 5′-AGATTTACAGGCTTGGCACCA-3′ | (SEQ ID NO: 33) | 62 | |
| Litsty | PEN3-1F | 5′-CCTGGTCTTCTTGGCCTCC-3′ | (SEQ ID NO: 34) | 62 | 157 |
| PEN3-1 | PEN3-1R | 5′-TAGAGGGCACACGTTGCCC-3′ | (SEQ ID NO: 35) | 62 | |
| ALF | ALFF | 5′-GTGAACTCCGCTGTTAAAGAC-3′ | (SEQ ID NO: 36) | 62 | 507 |
| | ALFR | 5′-TACACCACACTTGTATATTACAG-3′ | (SEQ ID NO: 37) | 62 | |

The PCR mixtures are prepared according to the following final concentrations: 1 μM of primer mixture, 1 mM dNTP, 1.5 mM MgCl₂; buffer 10× to 1×; 0.5 μl Taq for 50 μl of final volume (QS H₂O). Each PCR reaction was carried out with an initial denaturation step of 15 min at 97° C., followed by amplification of the cDNA [denaturation at 97° C. for 1 min, hybridization at 62° C. for 1 min, elongation at 72° C. for 1 min (the number of cycles varies according to the gene amplified)], a final elongation step at 72° C. for 10 min, and storage at 4° C.

The PCR products for each gene, previously amplified, are purified using the “QIAquick PCR Purification Kit Protocol” kit (Qiagen) according to the supplier's recommendations.

The amplified and purified PCR products are assayed on a spectrophotometer (abs. 260 and 280 nm, ratio close to 1.8) in order to quantify them, and are then concentrated to 100 ng/μl for membrane deposition.

### Membrane Preparation

The purified and concentrated PCR products are denatured at 98° C. for 10 min.

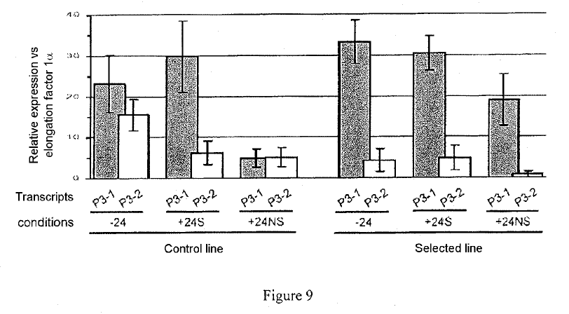

The PCR products (probe cDNA) are deposited on two nylon membranes in a proportion of 100 ng/spot, according to the scheme of the organization shown in FIG. 10.

Legend of FIG. 10:

### =ALF

[symbol] =crustin
◯=glycine-rich

### =Litsty PEN3-1

[symbol] =lysozyme [symbol]
=cysteine-rich.

The membranes are dried for 15 min at ambient temperature. The PCR products deposited on the membranes are again denatured using 0.5 M NaOH for 15 min (twice) by capillary action. The membranes are neutralized with 0.5 M Tris HCl, pH 7.4, for 10 min (twice), and then dried for 10 min at ambient temperature.

The final fixing of the PCR products is carried out under UV.

The membranes are subsequently dried with a view to storage in the dark at ambient temperature.

Preparation of Target cDNAs

A cDNA preparation was obtained from the total RNA isolated from the haemocytes either of a nonselected shrimp not surviving the infection, or a selected shrimp surviving the infection.

In order to obtain the cDNAs of each of the experimental conditions that will be hybridized on the membranes (target cDNAs), the cDNAs of each of the genes encoding the six antimicrobial peptides were amplified with the same pairs of specific primers and under the same PCR conditions as for the preparation of the probe cDNAs. On the other hand, digoxigenin-labelled dUTPs (dUTP-DIG) (“PCR DIG labelling mixture”, Roche Diagnostics) were introduced.

### Hybridization and Revealing of Membranes

In a first step, the membranes on which the probe cDNAs are fixed were prehybridized with a pre-hybridization buffer composed of 6×SSC, 0.1% N-lauroylsarcosine, 0.02% SDS, 1% skimmed milk and 1% Tween 20, for 45 min.

In a second step, the target cDNAs obtained were denatured at 98° C. for 10 min, and then plunged into ice. Each target cDNA was deposited onto its respective membrane, and incubated for 30 min at ambient temperature.

The membranes were subsequently washed twice with washing buffer W1 (0.1×SSC, 0.1% SDS) for 1 min with vigorous shaking.

The membranes were then incubated for 30 min at ambient temperature and with gentle shaking, with 450 μl of 1×TBS (10 mM Tris HCl, pH 7.4; 150 mM NaCl; 0.3% BSA), 1% skimmed milk, 2% dextran sulphate, containing a 1/5000 dilution of an anti-digoxigenin antibody (Fab) coupled to peroxidase.

The membranes were subsequently washed twice with 0.5 ml of 1×TBS for 1 min with vigorous shaking.

The membranes were incubated in 300 μl of revealing solution (TMB=3,3′,5,5′-tetramethyl-benzidine: colorimetric substrate for peroxidase) for 15 min in the dark.

The membranes were washed with sterile water (Dnase- and Rnase-free) for 5 min.

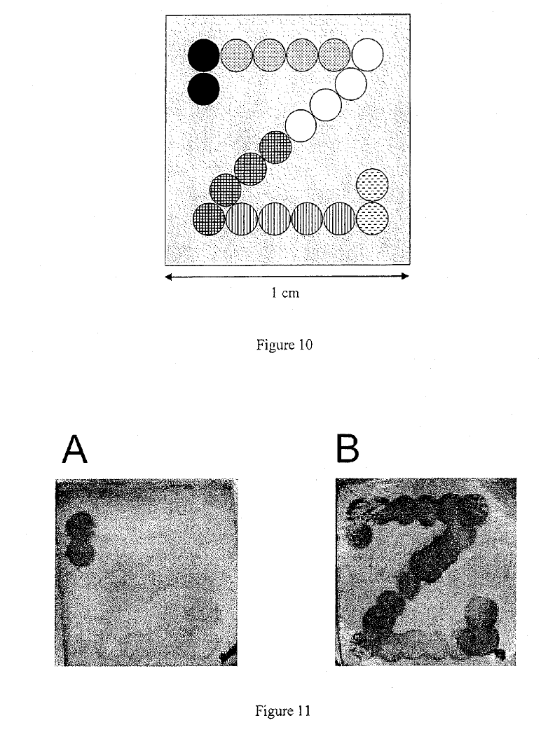

The results are given in FIG. 11.

Legend of FIG. 11:

A=membrane hybridized with the target cDNA originating from the nonselected and nonsurviving shrimp;
B=membrane hybridized with the target cDNA originating from the selected and surviving shrimp.

The results show that the hybridization signals that can be visualized on the membranes hybridized either with the target cDNA of a nonselected and nonsurviving shrimp (FIG. 11A) or with the target cDNA of a selected and surviving shrimp (FIG. 11B) show profiles that can be differentiated without any specific equipment.

In accordance with the quantitative PCR results, five of the peptides analysed (crustin, glycine-rich peptide, Litsty PEN3-1, cysteine-rich peptide, and lysozyme) exhibit variations in signals, with a greater intensity on the membrane hybridized with the target prepared from the selected shrimp surviving the infection (FIG. 11B) than on that hybridized with the target prepared from the nonselected shrimp not surviving the infection (FIG. 11A).

On the other hand, the ALF hybridization signals do not show any difference between the two membranes, and thus constitute a positive control for the experiment.

### Example 6

### Relationship Between the Basal Level of Expression of Antimicrobial Peptides and the Resistance Of Shrimp to Infection with Vibrio nigripulchritudo

L. stylirostris shrimp (56 individuals) were individually identified by injecting coloured silicone into an abdominal segment, and then a sample of haemolymph (200 μl) was taken from the ventral sinus in order to isolate the haemocytes therefrom. After 24 h of storing in tanks (conditions for recovery after stress), the shrimp were infected experimentally by injecting a solution containing “100 Vibrio nigripulchritudo bacteria” (60% lethal dose).

Individual monitoring of mortalities was carried out up to 96 h post-infection (theoretical end of mortalities) in order to distinguish the samples originating from shrimp having succumbed to the infection from the samples derived from those having survived: 43 individuals succumbed to the infection and 13 survived.

The basal level of transcription of the ALF, Litsty PEN3-1 and Litsty PEN3-2, Litsty PEN2-1 (PEN2), lysozyme, crustin, cysteine-rich-peptide and glycine-rich-peptide genes was measured by real-time PCR, as indicated above in Materials and Methods, in the samples taken 24 hours before the infection.

The quantification of the ALF, Litsty PEN2-1 (PEN2), lysozyme, crustin and cysteine-rich-peptide transcripts was carried out using the primers indicated in Table 1 above. In order to specifically quantify the transcription of Litsty PEN3-1 and of Litsty PEN3-2, the primers P3-1R and P3-1F for Litsty PEN3-1, and P3-2R and P3-2F for Litsty PEN3-2, described in Example 1, were used. The RNA of the glycine-rich protein was quantified using the primers SEQ ID NO: 43 (5′-GATGTTCCTGGCGGTGGTC-3′) and SEQ ID NO: 44 (5′-CGTTTTGCTCGGTGCAGTTC-3′). As in the previous experiments, the relative expression levels were standardized with respect to elongation factor 1α.

The expression of each of the transcripts was compared between the individuals having survived the infection (group 2) and those having not survived (group 1).

The results of the multivariate analysis (ANOVA test) of the expression data between the shrimp that were dead after infection (group 1) and the shrimp that survived the infection (group 2) are given in Table 3.

TABLE 3

| Gene name | Group 1 | Group 2 | Value p |
|---|---|---|---|
| Litsty PEN3-1 | 1.88 | 3.06 | 0.0054 |
| Glycine-rich | 1.23 | 1.02 | 0.0328 |
| protein | | | |
| Litsty PEN2 | 1.5 | 2.05 | 0.0407 |
| Litsty PEN3-2 | 11853.7 | 0.58 | 0.5083 |
| ALF | 1.05 | 1.63 | 0.03 |
| Lysozyme | 2.58 | 1.33 | 0.7344 |
| Cysteine-rich | 1.34 | 1.3 | 0.7971 |
| protein | | | |
| Crustin | 1.21 | 1.39 | 0.2523 |

In the case of Litsty PEN3-2, a very high inter-individual variability of the expression of this transcript is observed in group 1. The values observed in this group go from an absence of expression to a very strong expression, depending on the individuals. On the other hand, group 2 brings together only animals which very weakly express Litsty PEN3-2.

These results reveal significant differences between the surviving shrimp and the shrimp having not survived, and show that it is possible to distinguish statistically (p value<0.05) between the two groups of animals according to the levels of expression of Litsty PEN3-1, of the glycine-rich peptide, of Litsty PEN2 and of ALF, and also by the ratio of expression between Litsty PEN3-1 and Litsty PEN3-2.

Thus, a greater abundance of transcripts encoding Litsty PEN3-1, Litsty PEN2 and ALF is characteristic of shrimp capable of surviving the infection (mean relative expressions of: 3.06 vs. 1.88, 2.05 vs. 1.5 and 1.63 vs. 1.05, respectively), whereas a large abundance of transcripts encoding the glycine-rich peptide is characteristic of shrimp incapable of surviving the infection (mean relative expression of 1.23 vs. 1.02). Furthermore, the shrimp capable of surviving the infection have a higher ratio between Litsty PEN3-1 and Litsty PEN3-2 than the shrimp incapable of surviving the infection (1.6×10⁻⁴ for group 1, versus 5.3 for group 2).

1. Method for evaluating the capacity of resistance, of a population of penaeid shrimp, to infection with a pathogenic bacterium, characterized in that it comprises the quantification of the mRNA of the PEN3-1 isoform of penaeidin-3 in haemocytes taken from a sample of noninfected animals of the test population.

2. Method according to claim 1, characterized in that it also comprises the quantification of the mRNA of the PEN3-2 isoform of penaeidin-3, and the determination of the PEN3-1 isoform/PEN3-2 isoform ratio.

3. Method according to either one of claims 1 and 2, characterized in that it also comprises the quantification of at least one of the following RNAs:
the penaeidin-2 (PEN-2) mRNA;
the glycine-rich-protein mRNA;
the ALF mRNA.

4. Method according to either one of claims 1 and 2, characterized in that it is used to evaluate the capacity of resistance, of a line of penaeid shrimp, to infection with a pathogenic bacterium, and in that it also comprises the quantification, in haemocytes taken from the noninfected animals of the test line, of at least one of the following RNAs:
the lysozyme mRNA;
the penaeidin-3 mRNA.

5. Method according to claim 4, characterized in that it also comprises the comparison of the amount of one or more of said mRNAs with the average amount of the same mRNA(s) in noninfected animals of a control line of shrimp of the same species.

6. Method according to either one of claims 4 and 5, characterized in that it comprises the comparison between the amounts of mRNA of the two members of at least one of the following pairs:
lysozyme/penaeidin-3;
lysozyme/PEN3-2 isoform;
PEN3-1 isoform/penaeidin-3;
PEN3-1 isoform/PEN3-2 isoform.

7. Method for evaluating the outcome of an infection with a pathogenic bacterium in penaeid shrimp, characterized in that it comprises the quantification of the crustin mRNA in haemocytes taken from the test animal 6 to 72 hours after the infection.

8. Method according to claim 7, characterized in that it also comprises the comparison of the amount of crustin mRNA with the amount of the same mRNA in haemocytes taken from the test animal prior to the infection.

9. Method according to either one of claims 7 and 8, characterized in that it also comprises the quantification of the mRNA of the peptide rich in proline and in cysteine, in haemocytes taken from the test animal 6 to 72 hours after the infection, and, advantageously, the comparison of the amount of mRNA thus determined with the amount of the same mRNA in haemocytes taken from the test animal prior to the infection.

10. Method according to any one of claims 7 to 9, characterized in that it also comprises the quantification of one or more of the following mRNAs:
the lysozyme mRNA;
the penaeidin-3 mRNA;
the mRNA of the PEN3-1 isoform of penaeidin-3,
in haemocytes taken from the test animal 6 to 72 hours after the infection, and, advantageously, the comparison, for each mRNA chosen, of the amount thus determined with that of the same mRNA in haemocytes taken from the test animal prior to the infection.

11. Method according to any one of claims 7 to 9, characterized in that it also comprises the quantification of the penaeidin-2 mRNA in haemocytes taken from the test animal 6 to 72 hours after the infection, and, advantageously, the comparison of the amount thus determined with that of the same mRNA in haemocytes taken from the test animal prior to the infection.

12. Method according to any one of claims 1 to 11, characterized in that the penaeid shrimp belong to the species L. stylirostris.

13. Method according to any one of claims 1 to 12, characterized in that the infection is a vibriosis.

14. Kit of reagents for carrying out a method as defined in any one of claims 1 to 13, in which the quantification of the transcripts is performed by quantitative RT-PCR, said kit comprising, in addition to the buffers and reagents necessary for the amplification of the reaction and for the detection of the amplification products, at least one pair of primers specific for each of the mRNAs to be assayed.

15. Kit of reagents according to claim 14, characterized in that it comprises a pair of primers specific for the mRNA of the PEN3-1 isoform of penaeidin-3, and a pair of primers specific for the mRNA of the PEN3-2 isoform of penaeidin-3.

16. Kit of reagents according to claim 15, characterized in that it also comprises one or more pair(s) of primers chosen from:
a pair of primers specific for the penaeidin-2 (PEN-2) mRNA;
a pair of primers specific for the glycine-rich-protein mRNA;
a pair of primers specific for the ALF mRNA;
a pair of primers specific for the lysozyme mRNA.

17. Kit of reagents according to any one of claims 14 to 16, characterized in that it comprises a pair of primers specific for the crustin mRNA, and a pair of primers specific for the mRNA of the peptide rich in proline and cysteine.

18. DNA chip, characterized in that it comprises a probe specific for the mRNA of the PEN3-1 isoform of penaeidin-3, and a probe specific for the mRNA of the PEN3-2 isoform of penaeidin-3.

19. DNA chip according to claim 18, characterized in that it also comprises one or more probes chosen from:
a probe specific for the penaeidin-2 (PEN-2) mRNA;
a probe specific for the glycine-rich-protein mRNA;
a probe specific for the ALF mRNA;
a probe specific for the lysozyme mRNA.

20. DNA chip according to either one of claims 18 and 19, characterized in that it also comprises a probe specific for the crustin mRNA, and/or a probe specific for the mRNA of the peptide rich in proline and cysteine.


SEQUENCE LISTING (45 sequences)

SEQ ID NO: 1 - DNA, 226 bp, Litopenaeus stylirostris
gcttatttatcaaccattacaacgtctgcacgtcagctgtgttttgcaattctgttatttgcatttcaatgttgtataaattcatacatcatgatatacaggcaatcattgtttattaaccccaactaatactggaatcctggctttagcattgcattcgtattatttgcagtaataatggtaaccctggtgccaagcctgtaaatctagaacatagagctcgaag

SEQ ID NO: 2 - DNA, 294 bp, Litopenaeus stylirostris
ggttccctcctccgtccgccatgcgcctcgtggtctgcctggtcttcttggcctccttcgccctggtctgccaaggccaaggctacaagggcggttacacaggcccggtagtcaggcccttcgtgagaccgataggcaggcccttcgtgacaccgataggcaggcctgttgtttcgggcaacgtgtgccctctatcgtgccggggcattaccaccttacaagcgcgttcttgctgcagccgcttggggcgctgctgtcgcgaggccaaggggtactccggctgatggagaagac

SEQ ID NO: 3 - DNA, 270 bp, Litopenaeus stylirostris
ggttccctcctccgtccgccatgcgcctcgtggtctgcctggtcttcttggcctccttcgccctggtctgccaaggccaaggctacaagggcggttacacaggcccggtagtcaggcccttcgtgagaccgataggcaggcctgttgtttcatacaacgtgtgccctctatcgtgccggggcattaccaccttacaagcgcgttcttgctgcagccgcttggggcgctgctgtcgcgaggccaaggggtactccggctgatggagaagac

SEQ ID NO: 4 - DNA, 632 bp, Litopenaeus stylirostris
tcccggggcggtttagtgtaggtggtggcggtttaggtgtaggtggtggcttcccggggcggtgtaggtgtaggtggcggtcttggcgtgggaggaggccttggagttagcggtggctcaagcaactgcaggtattggtgcaagactccggagggtcaagcctactgctgcgagtcggcccacgaaccagagacacctgttggcaccaagctactcgactgcccccaagtccgtcccacatgcccacgcttccatgggccccccacgacctgttccaacgactacaagtgtgctggcctcgacaagtgttgcttcgacaggtgtctgggagaacacgtgtgcaagcctccttcggccttcggacagcaagttttcggatgaagaataaacacaaaaggattttaaaggatgaagagaaagacgaaaagaccatctgaaggacgaccgatgttttggaatttgactgaaaaaagaaagaaaaactaacagggaattctttctttctgtaggatttatctgattaacatgatttttgttatatgttaattagactgtattctgtcaaaagaaacttatagagaaaaaaaaaaaaaaaaaaaaaaaagcttgtacacctcggccgcgaccacgct

SEQ ID NO: 5 - DNA, 551 bp, Litopenaeus stylirostris
tctaatacgactcactatagggctcgagcggccgcccgggcaggtccagacgctgttctgctcctgtgtggtggccgaactctggacgcatccctgctgccatcatgaagacctacagtcgggtctccgtctttgtcttattggttgcgatcttgcacacgtcacaaggatcttccttttcacctcccagaggacctccgggctggggacctccatgcgtacaacaaccgtgccctaagtgcccatatgatgattacaagtgtccgacgtgtgataaattcccggagtgtgaggagtgcccccatattagtataggatgtgaatgcggctattttagctgcgaatgtccgaagcctgtgtgtgaaccgtgcgagagtcccatcgccgagttgataaaaaagggaggttataaaggataaagaagcgagaacgaagccacagcattctttcacaaacctcactgaactgaaagaagagagagggagagaggggggagggagagggagagagagagagaatagaacgtgagatgggagaaaggagagatgttt

SEQ ID NO: 6 - DNA, 20 bp, Artificial
  Amorce PCR
ggttccctcctccgtccgcc

SEQ ID NO: 7 - DNA, 20 bp, Artificial
  Amorce PCR
gtcttctccatcagccggag

SEQ ID NO: 8 - DNA, 24 bp, Artificial
  Amorce PCR
accgataggcaggcccttcgtgac

SEQ ID NO: 9 - DNA, 22 bp, Artificial
  Amorce PCR
cgatagagggcacacgttgccc

SEQ ID NO: 10 - DNA, 20 bp, Artificial
  Amorce PCR
gagaccgataggcaggcctg

SEQ ID NO: 11 - DNA, 22 bp, Artificial
  Amorce PCR
cgatagagggcacacgttgtat

SEQ ID NO: 12 - DNA, 19 bp, Artificial
  Amorce PCR
gccactcctgcacctacaa

SEQ ID NO: 13 - DNA, 21 bp, Artificial
  Amorce PCR
ttcacaacaccggatttgctg

SEQ ID NO: 14 - DNA, 19 bp, Artificial
  Amorce PCR
ccatgcgcctcgtggtctg

SEQ ID NO: 15 - DNA, 22 bp, Artificial
  Amorce PCR
gaacgcgcttgtaaggtggtaa

SEQ ID NO: 16 - DNA, 19 bp, Artificial
  Amorce PCR
ccatgcgcctcgtggtctg

SEQ ID NO: 17 - DNA, 21 bp, Artificial
  Amorce PCR
agcaattgcggcatctgggaa

SEQ ID NO: 18 - DNA, 19 bp, Artificial
  Amorce PCR
cgtctgcacgtcagctgtg

SEQ ID NO: 19 - DNA, 20 bp, Artificial
  Amorce PCR
ggcttggcaccagggttacc

SEQ ID NO: 20 - DNA, 19 bp, Artificial
  Amorce PCR
ccaagtccgtcccacatgc

SEQ ID NO: 21 - DNA, 20 bp, Artificial
  Amorce PCR
ctgtcgaagcagcacttgtc

SEQ ID NO: 22 - DNA, 19 bp, Artificial
  Amorce PCR
gccctaagtgcccatatga

SEQ ID NO: 23 - DNA, 19 bp, Artificial
  Amorce PCR
gccgcattcacatcctata

SEQ ID NO: 24 - DNA, 21 bp, Artificial
  Amorce PCR
ggtgctggacaagctgaaggc

SEQ ID NO: 25 - DNA, 25 bp, Artificial
  Amorce PCR
cgttccggtgatcatgttcttgatg

SEQ ID NO: 26 - DNA, 19 bp, Artificial
  Amorce PCR
tggtggccgaactctggac

SEQ ID NO: 27 - DNA, 20 bp, Artificial
  Amorce PCR
ctcctttctcccatctcacg

SEQ ID NO: 28 - DNA, 20 bp, Artificial
  Amorce PCR
tgcaccgagcaaaacgtcag

SEQ ID NO: 29 - DNA, 22 bp, Artificial
  Amorce PCR
tcgaggaattcacactgaaaca

SEQ ID NO: 30 - DNA, 20 bp, Artificial
  Amorce PCR
gtggtggcggtttaggtgta

SEQ ID NO: 31 - DNA, 20 bp, Artificial
  Amorce PCR
catcggtcgtccttcagatg

SEQ ID NO: 32 - DNA, 22 bp, Artificial
  Amorce PCR
tatcaaccattacaacgtctgc

SEQ ID NO: 33 - DNA, 21 bp, Artificial
  Amorce PCR
agatttacaggcttggcacca

SEQ ID NO: 34 - DNA, 19 bp, Artificial
  Amorce PCR
cctggtcttcttggcctcc

SEQ ID NO: 35 - DNA, 19 bp, Artificial
  Amorce PCR
tagagggcacacgttgccc

SEQ ID NO: 36 - DNA, 21 bp, Artificial
  Amorce PCR
gtgaactccgctgttaaagac

SEQ ID NO: 37 - DNA, 23 bp, Artificial
  Amorce PCR
tacaccacacttgtatattacag

SEQ ID NO: 38 - protein, 87 aa, Litopenaeus stylirostris
MRLVVCLVFLASFALVCQGQGYKGGYTGPVVRPFVRPIGRPFVTPIGRPVVSGNVCPLSCRGITTLQARSCCSRLGRCCREAKGYSG

SEQ ID NO: 39 - protein, 79 aa, Litopenaeus  stylirostris
MRLVVCLVFLASFALVCQGQGYKGGYTGPVVRPFVRPIGRPVVSGNVCPLSCRGITTLQARSCCSRLGRCCREAKGYSG

SEQ ID NO: 40 - DNA, 1126 bp, Litopenaeus  stylirostris
  misc_feature (2)..(2): n is a, c, g, or t
cncatatgatttattttgactgatagtgacctgttcgttgcaacaaattgatgagcaatgcttttttataatgccaactttgtacaaaaaagttgggtcgtcaccagtgccattatatacctcaaaatctacaatgggtttgagttcaactttcgtgagtgccgtgttggttgtggcactggtggcacccttagcgcccccatgccacgggttttcgctaaaagacctttttgtacctgtcattaaggaccaagtctctgatctgtggagaaccggagacattgacctcgtcggccactcctgcacctacaatgtcaagccagatatccaaggctttgaactctacttcatcgggtctgtcacgtgcccgggctggaccaccctccggggagagtccaacacccgcagcaaatccggtgttgtgaactccgctgttaaagacttcatccagaaggctctcaaggcaggtctggtcaccgaagaagaagccaaaccacaccttgtataataccatagaaatagggagagaggagggggtcatttattttttccttagtcaccccttgatttactcacgttttctatggtcgtctagtaggtctaattcatacattaattaatatctatattggttgtgttacccacctatgtaatggtatatttcttttatttttatgtaccttttcattttattttgttttcatgattttatactgttacctctgaactactgctaggagttttctagacatatttaactgttttttgtaaacttttgagaaactaaaacgaatgatatatgatatgtatacatgatcgtcatttaacatagattttcttcaaagatacaagaaaatgcaagttgattatagaagcaaatgttgaaatgnccaatgtcaaactatctgtaatatacaagtgtggtgtaaaataaattaaataaatnaattntatttaattgaaaaaaaaaaaaaaaaaaaaaancccaacttttnttgtanaaaagntggncattaaaaaaaagctttgcttatcaattttttggcaacgaaaagggtccttnttcngncaaaaanaaaatttttntttgnccccccngcttganttcccctaaaggggagtcnaa

SEQ ID NO: 41 - DNA, 246 bp, Litopenaeus  stylirostris
ttggtgctggacaagctgaaggccgagcgcgagcgcggtatcaccatcgacatcgccctgtggaagttcgagaccaacaggttctacgtgaccatcatcgatgccccaggccatcgtgatttcatcaagaacatgatcaccggaacgtcccaggctgactgcgccgtgctgatcgtcgctgccggtaccggcgagttcgaggctggtatctcgaagaacgggcagacccgcgagcacgtgttgctg

SEQ ID NO: 42 - DNA, 621 bp, Litopenaeus  stylirostris
  variation (573)..(573): a ou t
tcgctgacatcgacacggtttctttcctgaagcaacgcagccatgtcgcgtgtgatgttcctggcggtggtcgctgctttcgccgtctgtgccgaggcagcttcactgggctctgaaggggaactgcaccgagcaaaacgtcagttctcttggtcaagcgaccaaaatgtggagcaatacggtggacaagaaagcggcggagagcagtttgtcagttcccatggcactttcctgggcaacactcagaaggtcggccaactaggtgtcgggaacaacaacaagcagtttgtgcaccaaggcctctcgatcttcggacccagcaagcaggaagtggaacaggttgcgactggacgtggtttccagcaccagactgtcagccaaaagggtagcatctttggcgccagcagccagaaagttattcagaggggaggcactaaacagattgtgactggcaagtgagacgcggatccagcctaggctctgcattgttgttgaaactggttgactctattccttcatgatcaaagacttgtactggtatttgtgatgactgtttcagtgtgaattcctcgaaattaaattttcctttacaaaaaaaaaaaaaaaaaaaaaaaaagcttg

SEQ ID NO: 43 - DNA, 19 bp, Artificial
  Amorce PCR
gatgttcctggcggtggtc

SEQ ID NO: 44 - DNA, 20 bp, Artificial
  Amorce PCR
cgttttgctcggtgcagttc

SEQ ID NO: 45 - DNA, 321 bp, Litopenaeus stylirostris
ccgctgggctctccctcgagcctcacctgcagacaccgacgctccgagcccgggttccctcctccgtccgccatgcgcctcgtggtctgcctggtcttcttggcctccttcgccctggtctgccaaggcgaagcgtacaggggcggttacacaggcccgatacccaggccaccaccctatggaagaccaccgttaggacctgtttgcaaccattgctacagactcgccttcccagatgcccgcaattgctgcagcaggttcggacgttgttgtcacttagtaaaaggataaagaaattgatggagaagacaatggaaacct